(19) United States
(12) Patent Application Publication
(10) Pub. No.: US 2018/0049691 A1
(43) Pub. Date: Feb. 22, 2018

(54) Device for Monitoring the Perceived Pain Score

(71) Applicant: Heart Spòlka Z Ograniczona Odpowiedzialnoscia, Wroclaw (PL)

(72) Inventors: Radoslaw Nowosielski, Opole (PL); Mateusz Rulewicz, Wroclaw (PL); Jedrzej Baranski, Sycow (PL); Arkadiusz Jagodzinski, Wisznia Mala (PL)

(73) Assignee: Heart Spòlka Z Ograniczona Odpowiedzialnoscia, Wroclaw (PL)

(21) Appl. No.: 15/557,946

(22) PCT Filed: Mar. 17, 2016
(86) PCT No.: PCT/PL2016/000029

§ 371 Date: Sep. 13, 2017

(30) Foreign Application Priority Data
Mar. 20, 2015 (PL) P.411674

(51) Int. Cl. A61B 5/00; G06F 19/00

(52) U.S. Cl. CPC A61B 5/4824; A61B 5/0022; H02J 7/025; A61B 2560/0214; A61B 2560/0456; G06F 19/3418

(57) ABSTRACT

The present invention relates to a device for monitoring the perceived pain, particularly useful for creating documentation for the assessment of pain experienced by the patient in pain therapy. The device comprises a set of mobile devices in the form of remote controllers (1) placed on the charger (10) which are connected to the server (2). A server (2) on which data is stored in the database (11) is equipped with a user interface (12). Each remote controller (1) is equipped with a microcontroller (3), to which the remote communications unit (4), the remote controller buttons (6) and power supply of the remote control (7) are connected. It is possible to connect at least one light source (8), preferably LEDs, and/or a vibration motor (9) to the power system of the remote controller (7).

The present invention is a device for monitoring the perceived pain, particularly useful for creating documentation for the assessment of the pain experienced by the patient in pain therapy.

Computer tool for assessing pain, known from U.S. Pat. No. 8,046,241, is a computer, on the screen of which, in the first place, an image of the human body is presented and the patient, using the touch screen, interactively indicates the area of perceived pain, and then, on the second screen, where a minimum-to-maximum colour scale of pain intensity is presented, the patient moves the pointer to determine the intensity of pain and marks a box with a type of pain, which is used to indicate the type of pain and its depth. Upon completion of these two steps, the patient is asked to confirm the information about the pain. Patient data is processed by the computer together with the patient history, and the report can be accessed by a doctor.

The user interface for the assessment of pain, known from US patent application No. US20040267099, is based on a pain assessment scale and collects information about the pain of the patients and their mental health. The data is stored in the memory of the unit after it is entered by its operator. The interface is equipped, in the first place, with a device, wherein the input device comprises at least one keyboard and a touch screen, while the second input device comprises at least one keypad, a speech recognition unit, touch screen, pointing device, and a detector of the physiological condition of the patient, however, these devices may be the same. Further, it also includes an output device to provide the operator with at least one pain assessment scale and instructions on how to respond to pain assessment. The device may comprise a defibrillator and/or a patient monitoring device.

A similar device constituting a complex system with a pain assessment method is known from international patent application No. WO2002032304. A method of monitoring pain in a patient comprises the following steps: providing patient with a communication device, providing a data processor capable of communicating with the communication device of the patient with a microprocessor, providing a patient with a pain questionnaire for each of a series of measurement points in order to complete the questionnaire, transmitting the results of the questionnaire to the data processor, processing results by means of a data processor and thus monitoring the patient's pain. Provision of pain questionnaire is performed on each of a series of time points by a patient without any help from the medical staff. The pain questionnaire of the patient includes a request selected from a group consisting of a visual analogue scale of pain, visual analogue scale of mood, scale of scattered pain and pain relief scale, also in digitized form. Furthermore, the method comprises operating an effector function on the basis of the processed results from the pain questionnaire, which comprises administering analgesics used by the patient and the regulation of analgesia in order to form an output signal informing the medical personnel that the patient needs attention. The patient's pain management system comprises a communication device with a microprocessor, an effective pain questionnaire and data processor to automatically communicate with the communication device of the patient. Furthermore, the system comprises an effector device capable of communicating with a data processor, although the effector device is effective to perform effector functions. The patient's communication device is a hand-held device containing a touch screen and a microprocessor or a personal digital assistant.

Pain assessment known from the international patent application No. WO2003105686, is a sequence of collecting information about pain based on a mechanism in which data is collected from the patient, in particular information about pain or other afflicting ailments. Then, a profile of the patient's pain is generated and the pain characteristics are identified along with the pain mechanism responsible for the pain of the patient. Based on this analysis, a right treatment can be chosen.

The system and method for multidimensional evaluation of pain are known from the US patent application No. US20010037222. A method of quantifying the state of pain of the patient comprises the following steps: obtaining data on the patient's pain episode; assessing the patient's pain and generating a multidimensional assessment of pain determining the state of pain of the patient. Pain assessment stage involves many pain factors, which are selected from the group consisting of the emotions of the patient, movement of the patient, facial expression, verbal signals, the position of the patient and areas of observation of the patient. Furthermore, the pain assessment stage includes obtaining evident analgesic factors, which are selected from the group consisting of pain intensity, the mood of the patient, side effects of medications and pain. Then, a notice for intervention is generated when the multidimensional assessment of pain exceeds a certain level. A system for obtaining information about pain includes a central computer with software, database and memory, connected to any network of mobile computing devices such as PDAs, cell phones or similar devices.

Ruler to assess pain with a healthcare professional is known from Chinese Utility Model No. CN202699103. The ruler has six convex marks determining the level of pain, and on the other side of the ruler there is a description of pain corresponding to the symbol of pain.

The method of extracorporeal diagnosis of pain and anatomical abnormalities of the body and a device to implement this method are known from Polish patent No. PL212304. The method concerns a simultaneously carried measurement of interconnected planimetric and dynamic indicators, where using a white light, infrared, ultraviolet, electrical impedance, electric or magnetic field distribution in relation to the distribution of pressure and/or temperature and or blood flow in a foot and/or acceleration of the body, a complex first rank indicator for each foot is created, reflecting the anatomical and functional characteristics of both feet, and the first rank coefficients of the left and the right feet are weighted mathematically using the second rank coefficients evaluating the symmetry at rest relative to the reference population and/or functionally compared to previous results of the same patient. The device comprises two sectors, where one sector is used to perform measurements in the horizontal plane and consists of a transparent sheet with the pressure sensors in the bottom, in particular the front left, front right, rear left, rear right, and an imaging array or a scanner, as well as temperature sensors, microcirculation sensors, while the second sector for conducting measurement in the vertical plane comprises a camera to photograph the body, chest motion sensors, temperature sensors and microcirculation sensors.

The essence of the device according to the invention lies in the fact that it comprises a set of mobile devices in the form of remote controllers placed on the charger, which are connected to the server equipped with a user interface. Each remote controller is equipped with a microcontroller, to which a communication unit, remote controller buttons and power system of the remote controller are connected. Communication unit of the remote controller is connected with a server communication unit, preferably wirelessly.

Preferably, the supply system of the remote controller is equipped with a battery power source, most preferably inductively charged.

Preferably, the supply system of the remote controller is connected to at least one light source, preferably LEDs, and/or a vibration motor.

Preferably, a set of mobile devices is connected to a server through a base station, which comprises a microprocessor.

Preferably, each remote control of the set of mobile devices is equipped with a remote communication module, which is connected with the remote controller through a communication module, a microprocessor and a server communication unit with a unit for server communication.

Preferably, the housing of the remote has the body of the remote in the form of a rectangular tray, closed at the top by a cover with holes and the labelling with a pain scale. The bottom panel of the body of the remote comprises a handle, preferably of a lanyard.

Preferably, the labelling with the pain scale has convex pictograms, next to which, along one edge a pain scale in Braille is located, and along the other edge, a colour scale of pain.

Preferably, the charger has a body of a charger with alignment rails of the remote controllers in front and in the back of the body of the charger, along the line of symmetry, cut-outs for the inductive coils are made in the body of the charger and on both sides of these cut-outs spacers are arranged.

Preferably, on the alignment guides of the remote controllers the front panel is mounted, wherein in the bottom parts of the alignment guides of the remotes and in the front panel at least one corner cut-out is made, of which at least one has an area larger than the cross-section area of the remote controllers.

Preferably, to the bottom wall of the body of the charger a fastening element is mounted and a hole is cut out for the power outlet.

Preferably, telescopic rails are mounted along the rear side walls of the body, preferably with the rail holes.

The device for monitoring the perceived pain solves the problem of documenting pain monitoring in hospitals, automates the monitoring and allows for remote monitoring of patients, while digitizing data without involving third parties, which allows to keep extensive statistical data concerning pain. The patients are reminded about the moment when they should assess their pain level with light signals and vibration. The device comprises compact remote controllers that are ergonomically designed and tailored to the size of the palm of a patient. The remote controllers are charged wirelessly, and closed in a sealed, solid casing to make them more resistant to actions of third parties and help keep them clean in hospital conditions. The option to program the remotes to automatically switch into sleep mode greatly extends the operating time of battery power sources. Furthermore, the device is characterized by extremely simple operation, which greatly facilitates its used by both the staff and patients.

In the embodiment of the invention illustrated in the drawing,

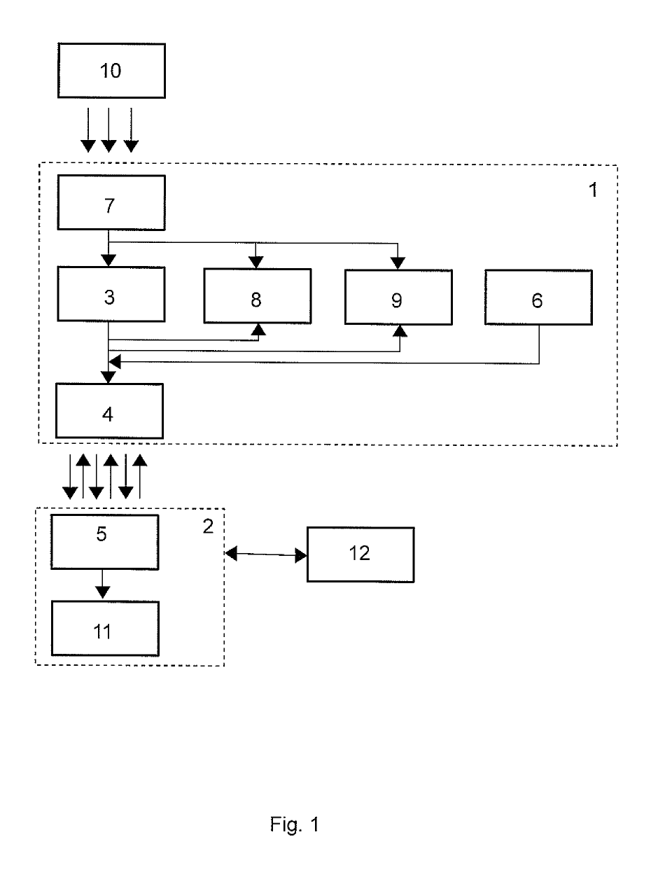

FIG. 1 shows a device for monitoring of the perceived pain,

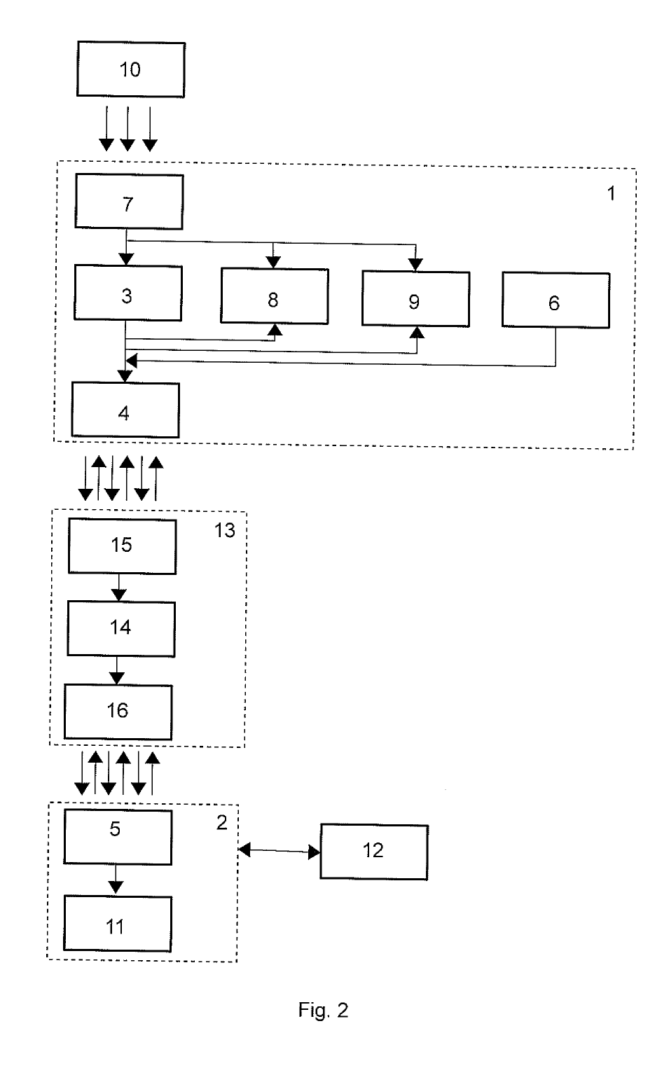

FIG. 2—a device for monitoring of perceived pain with a base station,

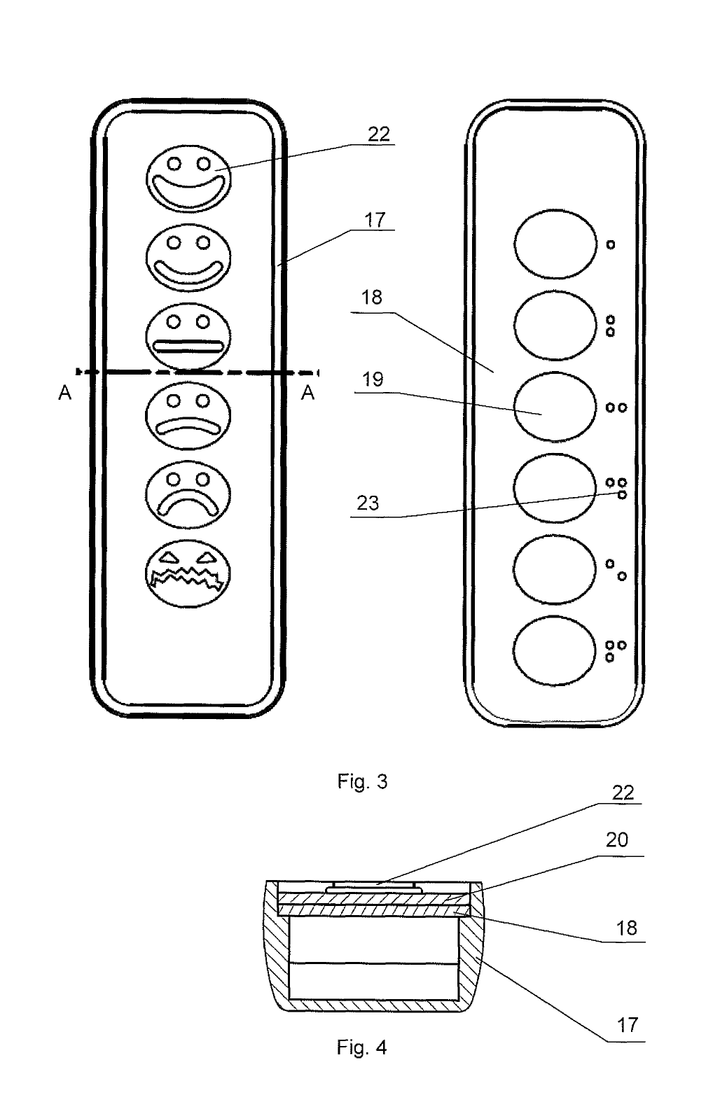

FIG. 3—body of the remote controller with and without the housing,

FIG. 4—A-A cross section of the remote controller

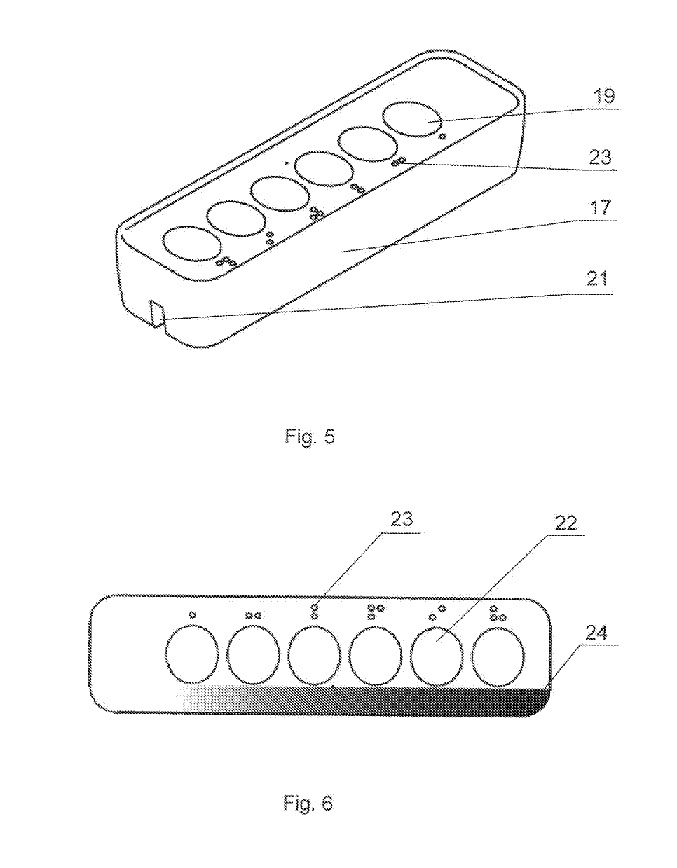

FIG. 5—casing of the remote controller in an axonometric view,

FIG. 6—front housing of the remote controller,

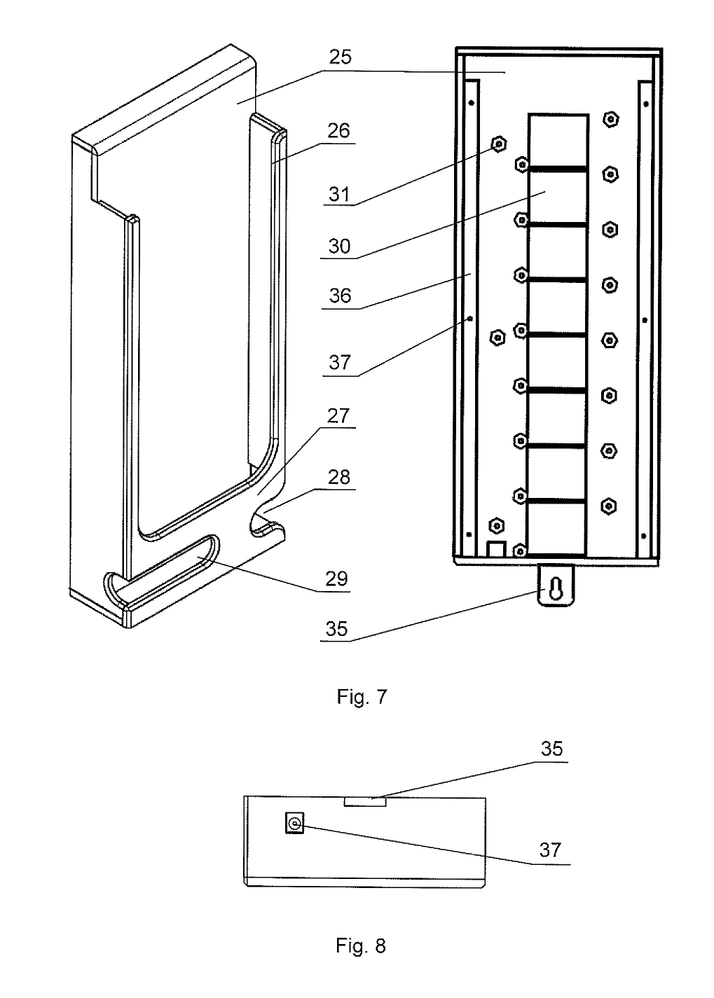

FIG. 7—body of the charger in front and rear view,

FIG. 8—the bottom panel of the body of the charger,

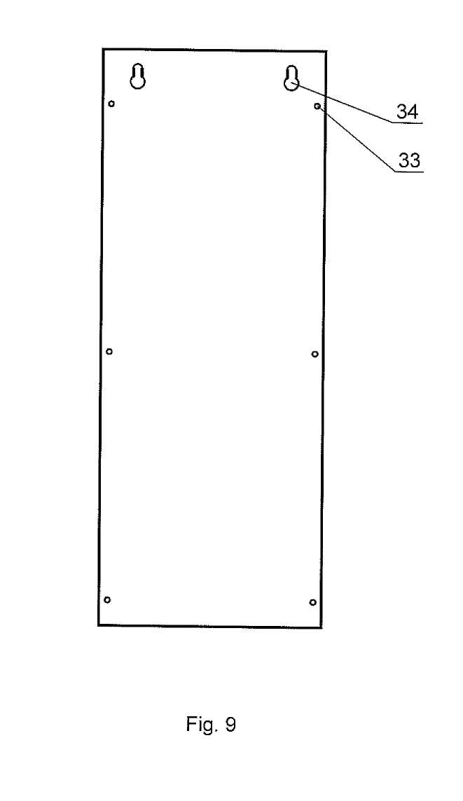

FIG. 9—rear panel of the housing of the charger and

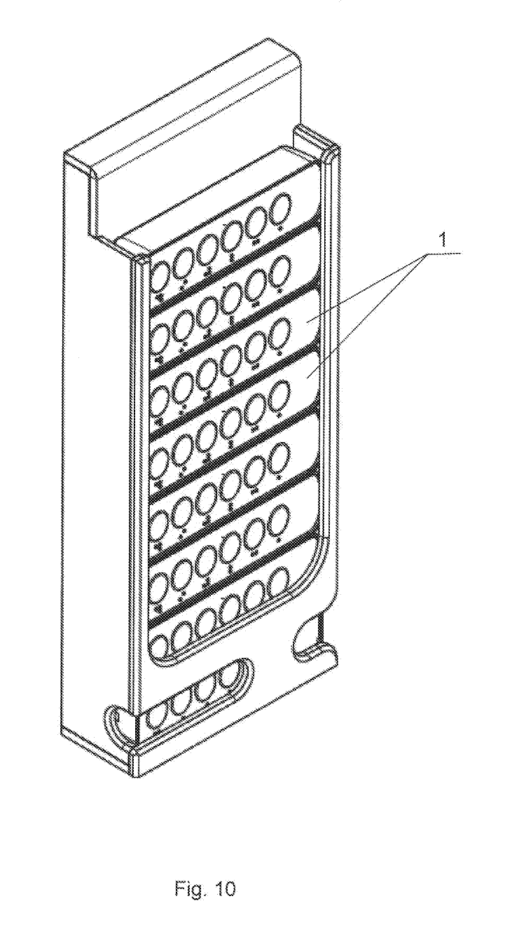

FIG. 10—the charger with the remote controllers.

### EXAMPLE NO. 1

The device for monitoring the perceived pain is a computer connected to the Internet, which in turn is connected to a set of six mobile devices—the remote controllers 1 placed on the charger 10. Remote controllers 1 are connected to the server 2 equipped with a user interface 12 in the form of a computer with a software and a database 11. Each remote controller 1 is equipped with a microcontroller 3, which is connected to a communication unit 4, buttons of the remote controller 6 and the supply system of the remote controller 7. The power supply system of the remote controller 7 is equipped with a battery power source, which is charged inductively. The communication unit of the remote controller 4 is connected to a server communication unit 5. Microcontroller 3 is connected to one light source 8 and a vibration motor 9. The housing of the remote controller constitutes a body of the remote 17 in the form of a rectangular tray closed at the top with a cover lid 18 with orifices 19 and the labelling with the pain scale 20. In the short side of the bottom of the body of the remote 17 there is a lanyard holder 21. The labelling with the pain scale 20 constitutes convex pictograms 22, located over the buttons of the remote controller 6, placed in the body of the remote controller 17 in the orifices of the cover 19; moreover, along the right edge of the labelling with the pain scale 20, there is a pain scale in Braille numbers 23, and along the left edge there is a colour pain scale 24. The charger 10 constitutes a body of the charger 25 with alignment guides for the remote controllers 26 in front. At the bottom of the alignment guides 26 there is a right corner cut-out 28. On the rear side of the body of the charger 25, along the symmetry line, there are cut-outs for the induction coils 30 and on both sides of these cut-outs 30 there are spacers 31, to which electronic circuits of the charger 10 are mounted. The side surface of the right corner cut-out 28 is larger than the cross-section area of the remote control 1. In the bottom of the body of the charger 25 a fastening element 35 is mounted and a hole for the power outlet 38 is cut.

### EXAMPLE NO. 2

A device for monitoring the perceived pain is made as in the first example except that the communication module of the remote controller 4 is connected wirelessly to a server communication module 5, a set of lights 8 in the form of fourteen white LEDs placed along two the longer sides of the body of the remote 17 is connected to the supply system 7. At the bottom, the alignment guides 26 are enclosed with front panel 27, also in the bottom of the alignment guides 26 and the front panel 27 there are two right corner cut-outs 28 and left corner cut-out 29 on the opposite side. The side surface of the left corner cut-out 29 is larger than the cross-section area of the remote controller 1. In addition, along the rear side walls of the body of the charger 25 there are mounting rails 36 with holes in the rail 37.

### EXAMPLE NO. 3

A device for monitoring the perceived pain is made as in the first or second example, except that a set of eight remote controllers 1 is connected to server 2 via a base station 13 which comprises a microprocessor 14, wherein each remote control 1 is equipped with a remote controller communication unit 4, which is connected via unit for communication with the remote controller 15, the microprocessor 14 and the unit of communication with the base station 13 that mediates communication between remote controllers 1 and a server 2. The server 2 stores patients' data in the database 11. A minicomputer is used to process information and handle all network-connected devices. Data received from remote controllers 1 is further transmitted through Ethernet using the http protocol and is stored on an external server 2. The communication also operates in the opposite direction—from the server 2 through the base station 13 to remote controllers 1. The remote controllers 1 send information on battery status, the remote controller buttons 6 and whether the battery power source is being charged in the charger 10 to the server 2. The server sends to the remote controllers 1 data on vibration settings and the intensity of the light sources 8, the mode in which the remote controllers 1 should operate and the time after which the remote controllers 1 should communicate again. Communication with the base station 13 is carried out in two cases—when any button 6 on the remote control 1 is pressed or when the time which was previously set by the server 2 has passed. The remote controller 1 initiates communication with the base station 13. Once connected, the remote controller 1 sends the following information: ID stored in the internal flash memory, the reading on the charging level of the battery power source, the number of the button 6 pressed and whether the remote controller 1 is currently being charged in the charger 10. Server 2 sends to the remote controller 1 information about the level of brightness of light sources 8, the level of vibration and whether the remote controller 1 should switch into active mode, in which it emits light and/or vibrates.

The purpose of the device for monitoring perceived pain is to inform patients in a medical facility, e.g. in a hospital that they should at a given time indicate the level of pain they perceive. This information is given by the alternate flashing of white LEDs and/or vibration of the vibrating motor 9 in the remote controller 1 of the patient. It signals the moment when the patient should press one of the six buttons 6 on the remote in order to inform about the pain they feel at that time. Information is then sent to the base station 13 through wireless communication, where it is further sent to the server 2, where it is stored in the database 11. The server 2 simultaneously calculates the time in which the subsequent measurement of the patient's pain takes place and sends the feedback information to the remote controller 1 via the base station 13. The remote controller 1 goes into sleep mode until the time indicated by the server 2. It is possible to send information about the perceived pain by pressing the button 6, even during the sleep mode of the remote controller 1. Also in this case, the above-described communication with the server 2 occurs. The remote controller 1 also constitutes a red LED that lights when there is no communication with the server 2, e.g. the remote controller 1 is out of reach, and during charging in charger 10. The charger 10 allows simultaneous charging of the eight remote controllers 1. It uses standard QI wireless charging.

Six monostable buttons 6 of the remote controllers are connected to inputs of the microcontroller 3. Information about pressing the remote controller button 6 is transmitted by radio to the base station 13. The housing of the remote controller 1 is sealed and cannot be opened again. The remote controller 1 does not constitute any charging port, hence the wireless charging system based on the Q1 standard has been used.

### LIST OF DESIGNATION ON THE DRAWING

1. remote controller,
2. server,
3. microcontroller,
4. communication unit of the remote controller,
5. communication unit of the server,
6. buttons of the remote controller,
7. power supply system of the remote controller,
8. light source,
9. vibration motor,
10. charger,
11. database,
12. user interface,
13. base station,
14. microprocessor,
15. unit for communication with the remote controller,
16. unit for communication with the server
17. body of the remote controller,
18. cover,
19. orifices in the cover,
20. labelling with the scale of pain,
21. lanyard holder,
22. convex pictograms,
23. pain scale in Braille numbers,
24. colour scale of pain,
25. body of the charger,
26. alignment rails for the remote controllers,
27. front panel,
28. right corner cut-out,
29. left corner cut-out,
30. cut-outs for the induction coils,
31. spacer,
32. rear panel,
33. mounting holes,
34. fastening holes,
35. fastening element,
36. mounting rails,
37. hole in the rail,
38. hole for the power outlet.

1. A device for monitoring the perceived pain is a computer connected to the Internet, which in turn is connected to mobile devices equipped with a pain scale, characterized in that it comprises a set of mobile devices in the form of remote controllers (1) placed on the charger (10), which are connected the server (2) with a user interface (12), wherein each remote control (1) is equipped with a microcontroller (3) to which a communication module of the remote controller (4), remote controller button (6), and a supply system of the remote controller (7) are connected.

2. A device according to claim 1, characterized in that the remote communication unit (4) is connected to the server communication unit (5), preferably wirelessly.

3. A device as claimed in claim 1, characterized in that the remote power supply circuit (7) is equipped with a battery power source.

4. A device as claimed in claim 3, characterized in that the battery power source is charged inductively.

5. A device as claimed in claim 1, characterized in that the power supply to the remote controller (7) is connected to at least one light source (8), preferably an LED.

6. A device as claimed in claim 1, characterized in that the power supply to the remote controller (7) is connected to the vibration motor (9).

7. A device as claimed in claim 1, characterized in that a set of mobile devices is connected to a server (2) via a base station (13), which comprises a microprocessor (14).

8. A device as claimed in claim 7, characterized in that each remote controller (1) of a set of mobile devices includes a communication unit of the remote control (4) which is connected via the communication unit with the remote controller (15), the microprocessor (14) and a unit for data communication with the server (16) with the unit for server communication (5).

9. A device as claimed in claim 1, characterized in that the housing of the remote comprises a body of the remote (17) in the form of a rectangular tray which at the top is closed with a cover (18) with the holes in the cover (19) and labeling with a pain scale (20).

10. A device as claimed in claim 9, characterized in that in the bottom wall of the body of the remote (17) a preferably lanyard holder is made (21).

11. A device as claimed in claim 9, characterized in that labeling with a pain scale (20) comprises convex pictograms (22) next to which, along the first edge, there is a pain scale written in Braille numbers (23) and along the second edge there is a color pain scale (24).

12. A device as claimed in claim 1, characterized in that the charger (10) comprises a body of the charger (25) which at the front has alignment guides for the remote controllers (26) and at the rear of body of the charger, (25) along a line of symmetry, in the body of the charger (25), there are cut-outs for the inductor coils (30) and on both sides of the cut-outs (30) there are spacers (31).

13. A device as claimed in claim 12, characterized in that on the alignment guides for the remote controllers (26), a front wall (27) is mounted, wherein at the bottom of the alignment guides for the remote controllers (26) and of the front wall (27) there is at least one corner cut-out (28, 29) wherein at least one has a greater surface that the cross-sectional area of the remote controller (1).

14. A device as claimed in claim 12, characterized in that in the bottom wall of the body of the charger (25) a fastening element (35) is fixed and a hole for the power outlet (38) is cut out.

15. A device as claimed in claim 12, characterized in that along the rear side walls of the body of the chargers (25) mounting rails (36) are installed, preferably with perforated rail (37).
